(19) United States
(12) Patent Application Publication
(10) Pub. No.: US 2021/0115509 A1
(43) Pub. Date: Apr. 22, 2021

(54) BIOMARKER FOR IDENTIFYING SPECIFIC EXPOSURE TO KETONES AND METHOD OF IDENTIFICATION USING THE SAME

(71) Applicant: KOREA INSTITUTE OF SCIENCE AND TECHNOLOGY, Seoul (KR)

(72) Inventors: Jae Chun RYU, Seoul (KR); YOON CHO, Seoul (KR)

(21) Appl. No.: 16/703,619

(22) Filed: Dec. 4, 2019

(30) Foreign Application Priority Data
Oct. 16, 2019 (KR) 10-2019-0128341

(51) Int. Cl. C12Q 1/6874; C12N 15/10

(52) U.S. Cl. CPC C12Q 1/6874; C12N 15/1096; C12N 15/1003

(57) ABSTRACT

Disclosed are a biomarker for identifying specific exposure to ketones, which are one of environmentally harmful substances that may be exposed in the indoor atmosphere environment, and a method for identifying exposure to ketones using the same. More specifically, disclosed are a biomarker composition and a kit for identifying specific exposure to ketones containing genes, the expression of which is increased or decreased specifically in human bronchial epithelial cell lines by exposure to five kinds of ketones including acetone, butanone, pentanone, hexanone and heptanone, and a method for identifying exposure to ketones using the same. Fast-throughput sequencing using a biomarker including a gene showing a changed expression pattern upon exposure to five types of ketones including acetone, butanone, pentanone, hexanone and heptanone is useful for rapidly and accurately monitoring and determining the contamination level of ketones in the environment.

### CROSS-REFERENCE TO RELATED APPLICATION

This application claims, under 35 U.S.C. § 119(a), the benefit of priority to Korean Patent Application No. 10-2019-0128341, filed on Oct. 16, 2019, the entire contents of which are incorporated herein by reference.

### BACKGROUND

### Technical Field

The present invention relates to a biomarker for identifying specific exposure to ketones and a method for identifying exposure to ketones using the same. More specifically, the present invention relates to a biomarker composition and a kit for identifying specific exposure to ketones containing genes, the expression of which is increased or decreased specifically in human bronchial epithelial cell lines by exposure to five kinds of ketones including acetone, butanone, pentanone, hexanone and heptanone, and a method for identifying exposure to ketones using the same.

### Background Art

Ketones are mVOCs (microbial volatile organic compounds) that are generated during the metabolism of microorganisms such as fungi and bacteria in the indoor environment, and are often reported to be one of substances exposed to the indoor environment (Schenkel D et al., A meta-analysis approach for assessing the diversity and specificity of belowground root and microbial volatiles, Front. Plant Sci., 6:707, 2015).

Exposure to ketones is known to mainly occur through respiration, thus irritating the respiratory system as well as the eyes and skin. Ketones have been reported to cause symptoms such as headaches, dizziness, nausea and fatigue (Korpi A., Microbial Volatile Organic Compounds. Crit. Rev. Toxicol., 39(2):139-193, 2009). Despite the potential risk of ketones to humans as described above, specific risk assessment data of ketones including acetone are not sufficient, and the detection methods for exposure are also limited to traditional methods such as GC-MS (gas chromatography-mass spectrometer) or HPLC (high-performance liquid chromatography). Quantification is possible using GC-MS or HPLC methods, but appropriate conditions for analysis should be determined and expensive equipment are required. Thus, it is an important task to find and utilize molecular indicators that can detect risks and gene expression in the human body through rapid assessment of risk using faster and simpler screening methods such as real-time polymerase chain reaction (RT-PCR) using primers, DNA microarray chips, or next-generation sequencing (NGS) and thereby to take and implement appropriate measures for exposure to ketones.

Next-generation sequencing (NGS) is an analysis method including dividing the genome into a number of fragments and combining the respective sequences to decipher the genome. After the human genome project in 2004, information of base sequences of a subject was first deciphered using Sanger's method (Ley et al., DNA sequencing of a cytogenetically normal acute myeloid leukaemia genome, Nature, 456 (7218): 66-72, 2008). Subsequently, in 2008, it became possible to decipher the base sequence at 1% of the cost of the initial sequencing technology (Wheeler D A et al., The complete genome of an individual by massively parallel DNA sequencing, Nature, 452: 872-876, 2008). RNA sequencing (RNA-seq) is a method that can analyze a transcriptome using NGS technology and obtain a variety of information about certain exons constituting transcripts that have been expressed by sequencing an RNA sequence expressed in a specific sample. Analysis is performed to compare expression levels of genes, or RNA sequencing is performed for analysis and integrated interpretation regarding gene structures such as SNP, In/Del and alternative splicing.

Recently developed deep sequencing technology is being used for RNA sequencing. Generally, one RNA population (all or poly(A)) is converted into a library of cDNA fragments provided with an adapter at one or both ends. Respective molecules, which are amplified or not, are sequenced in a highly efficient manner to obtain short sequences from one end (single-end sequencing) or short sequences from both ends (paired-end sequencing). The length of the sequences obtained is generally 30 to 400 bp, and depends on the sequencing technique used. Currently, technologies such as Illumina IG, Applied Biosystems SOLID, and Roche 454 Life Science systems have been applied to RNA sequencing (RNA-seq). The resulting reads obtained after sequencing are aligned to standard genomes or standard transcripts, or are de novo assembled in the absence of genome sequences to form a transcription map of the genome unit based on the transcriptional structure and expression level of each gene. Research on toxicogenomics which is a high-tech method using RNA sequencing technology combined with microarray techniques has brought about high-throughput analysis and quantitative analysis of expression patterns of genes expressed in specific tissues or cell lines through all chemicals including representative environmental pollutants as well as pharmaceuticals and new drug candidates. Accordingly, analysis on the frequency of expression of specific genes in specific cells identifies genes related to the adverse effects of drugs and harmful effects of environmental pollutants. This provides understanding of the harmful actions of environmental pollutants, the actions of drugs and the molecular mechanisms caused by the side effects of drugs, and enables searching for and identification of substances that cause toxicity and side effects.

### PRIOR ART LITERATURE

### Non-Patent Document

(Non-patent Document 1) Schenkel D et al., Front. Plant Sci., 6:707, 2015
(Non-patent Document 2) Korpi A., Crit. Rev. Toxicol., 39(2):139-193, 2009
(Non-patent Document 3) Ley et al., Nature, 456(7218):66-72, 2008
(Non-patent Document 4) Wheeler D A et al., Nature, 452:872-876, 2008

The above information disclosed in this Background section is provided only for enhancement of understanding of the Background of the invention and therefore it may contain information that does not form the prior art that is already known in this country to a person of ordinary skill in the art.

### SUMMARY OF THE DISCLOSURE

The present inventors obtained gene expression profiles of five types of ketones, specifically acetone, butanone, pentanone, hexanone and heptanone, from BEAS-2B human bronchial epithelial cell lines using NextSeq500 (Illumina, Inc., USA) and analyzing the same to find genes, the expression of which is commonly changed by exposure of ketones, and establishing a biomarker capable of detecting gene expression upon exposure to ketones and a method for identifying exposure using the same, thereby completing the present invention.

It is an object of the present invention to provide a biomarker composition for identifying specific exposure to ketones containing genes the expression of which is increased or decreased specifically in human bronchial epithelial cell lines by exposure to five kinds of ketones including acetone, butanone, pentanone, hexanone and heptanone, a kit for identifying specific exposure to the five types of ketones, and a method for identifying exposure to ketones using a genetic biomarker causing a specific expression change upon exposure to the five types of ketones.

The object of the present invention is not limited to those described above. The object of the present invention will be clearly understood from the following description and can be implemented by the means defined in the claims and combinations thereof.

In one aspect, the present invention provides a biomarker composition for identifying specific exposure to five types of ketones including acetone, butanone (2-butanone), pentanone (2-pentanone), hexanone (2-hexanone) and heptanone (2-heptanone), including an ADRA2A (adrenoceptor alpha 2A) gene of Gene Accession No. NM_000681 (GenBank).

The biomarker composition may include one or more of genes further selected from the following group: Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

Expression of the ADRA2A (adrenoceptor alpha 2A) gene of Gene Accession No. NM_000681 (GenBank) may be increased or decreased by exposure to five kinds of ketones including acetone, butanone, pentanone, hexanone and heptanone.

In another aspect, the present invention provides a kit for identifying specific exposure to five kinds of ketones including acetone, butanone, pentanone, hexanone and heptanone, the kit including an agent for measuring an expression level of a gene ADRA2A (adrenoceptor alpha 2A) of Gene Accession No. NM_000681 (GenBank).

The kit may further include an agent for measuring an expression level of a gene further selected from the following group: Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

The kit may include at least one of a reaction reagent, a labeling reagent or a wash buffer, wherein the reaction reagent is selected from the group consisting of a buffer used for hybridization, oligo (dt) beads for separating mRNA from total RNA, a reverse transcriptase for synthesizing cDNA from RNA, dNTPs, rNTP (premixed or separately fed), a ligase and a combination thereof.

The kit may further include human bronchial epithelial cells.

The agent for measuring the expression level of the gene may include sense and antisense primers or probes that complementarily bind to mRNA of the gene.

In another aspect, the present invention provides a method for identifying exposure to five ketones including acetone, butanone, pentanone, hexanone and heptanone, including a) isolating total RNA from somatic cells of each of experimental and normal control groups suspected of exposure to five ketones including acetone, butanone, pentanone, hexanone and heptanone, b) converting the total RNA of the experimental and control groups of step a) to cDNA by treatment with oligo-dT primers and reverse transcriptase to establish a cDNA library, c) sequencing the cDNA library obtained in step b) through high-throughput sequencing using next-generation sequencing (NGS) equipment, and d) determining an expression level of genes included in the composition of claim 1 or 2 in the analyzed data of step c) compared to a control group.

The somatic cells of step a) may include human bronchial epithelial cells.

The human bronchial epithelial cells may include BEAS-2B cells.

Other aspects and preferred embodiments of the invention are discussed infra.

### BRIEF DESCRIPTION OF THE DRAWINGS

The above and other features of the present invention will now be described in detail with reference to certain exemplary embodiments thereof, illustrated in the accompanying drawings, which are given hereinbelow by way of illustration only, and thus are not limitative of the present invention, and wherein:

FIG. 1a to FIG. 1e is a graph showing the result of analysis of the cytotoxicity of BEAS-2B, a human bronchial epithelial cell line, to five ketone compounds including acetone, butanone, pentanone, hexanone and heptanone;

FIG. 2 shows the overall gene expression behavior in human bronchial epithelial cells exposed to five ketones including acetone, butanone, pentanone, hexanone and heptanone using high-throughput sequencing; and

FIG. 3 shows the result of analysis of gene expression of 30 types of genes, the expression of which is changed 2.0 times or more in common in human bronchial epithelial cells exposed to five ketones including acetone, butanone, pentanone, hexanone and heptanone using high-throughput sequencing.

### DETAILED DESCRIPTION

The objects described above, as well as other objects, features and advantages of the present invention, will be clearly understood from the following preferred embodiments with reference to the attached drawings. However, the present invention is not limited to the embodiments, and may be embodied in different forms. The embodiments are suggested only to offer a thorough and complete understanding of the disclosed context and to sufficiently inform those skilled in the art of the technical concept of the present invention.

Unless the context clearly indicates otherwise, all numbers, figures and/or expressions that represent ingredients, reaction conditions, polymer compositions and amounts of mixtures used in the specification are approximations that reflect various uncertainties of measurement occurring inherently in obtaining these figures, among other things. For this reason, it should be understood that, in all cases, the term “about” should be understood to modify all numbers, figures and/or expressions. In addition, when numerical ranges are disclosed in the description, these ranges are continuous and include all numbers from the minimum to the maximum including the maximum within each range unless otherwise defined. Furthermore, when the range refers to an integer, it includes all integers from the minimum to the maximum including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include numbers such as 10%, 11%, 12% and 13% as well as 30%, and any sub-ranges of 10% to 15%, 12% to 18%, or 20% to 30%, as well as any numbers, such as 10.5%, 15.5% and 25.5%, between appropriate integers that fall within the range.

Also, the terms and abbreviations used herein may be interpreted as meanings commonly understood by those skilled in the art to which the present invention pertains unless otherwise defined.

Hereinafter, the present invention will be described in detail.

The present invention provides a biomarker composition and a kit for identifying specific exposure to ketones containing genes the expression of which is increased or decreased specifically in human bronchial epithelial cell lines by exposure to five kinds of ketones including acetone, butanone, pentanone, hexanone and heptanone, and a method for identifying exposure to ketones using the same.

One aspect of the present invention provides a biomarker composition for identifying specific exposure to five types of ketones including acetone, butanone (2-butanone), pentanone (2-pentanone), hexanone and heptanone (2-heptanone) including an ADRA2A (adrenoceptor alpha 2A) gene of Gene Accession No. NM_000681 (GenBank).

The biomarker is a substance that is capable of identifying exposure to five types of ketones including acetone, butanone, pentanone, hexanone and heptanone, and includes a gene exhibiting an increase or decrease in expression in an experimental group exposed to ketones, compared to a control group not exposed to the ketones.

The ketones are microbial volatile organic compounds (mVOCs) generated during the metabolism of microorganisms such as mold and bacteria in the indoor environment, are reported as one of the substances that are frequently exposed in the indoor environment, and are known to cause symptoms such as headaches, dizziness, nausea and fatigue.

In the present invention, the biomarker is a gene the expression of which is increased or decreased by 2.0 times or more, and the expression is specifically changed by five types of ketones including acetone, butanone, pentanone, hexanone and heptanone. Based on this feature, exposure to the five ketones can be identified.

The biomarker showing an expression change specifically depending on the degree of exposure to the five types of ketones may be an ADRA2A (adrenoceptor alpha 2A) gene of Gene Accession No. NM_000681 (GenBank).

In addition, a biomarker that specifically shows an expression change depending on the level of exposure to the five ketones may include, but is not limited to, one or more of genes further selected from the following group:

Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), Gene Accession No. (GenBank) NR_026693 (ZNF487, zinc finger protein 487).

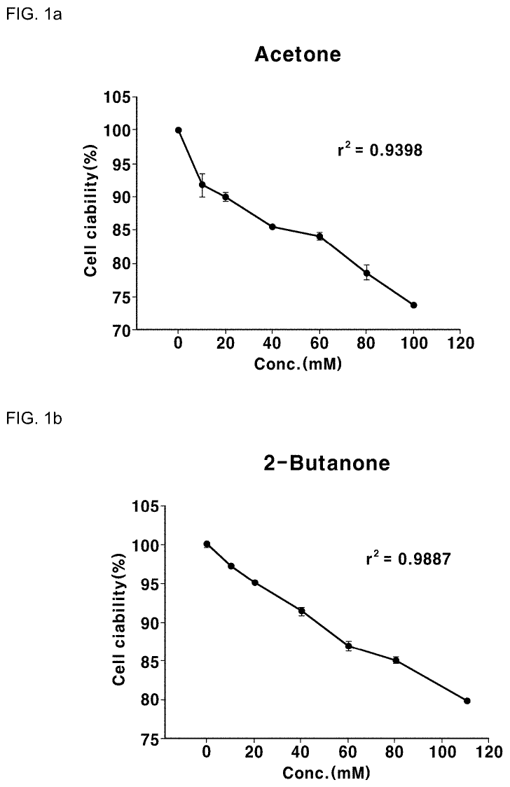

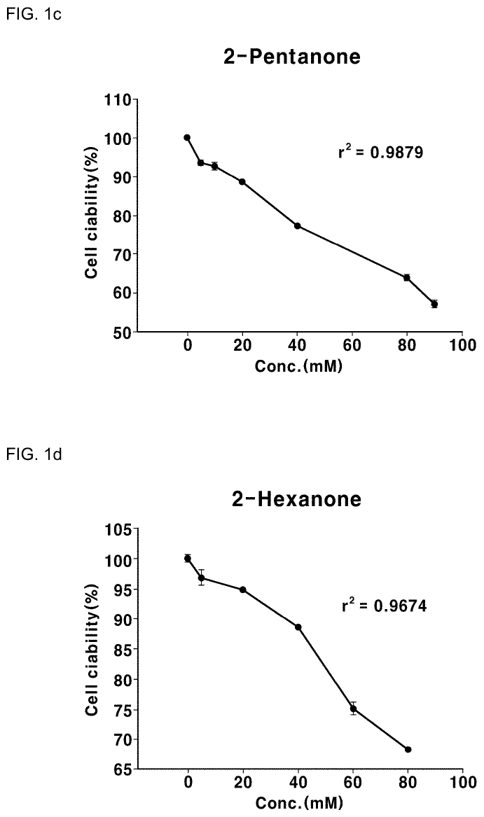

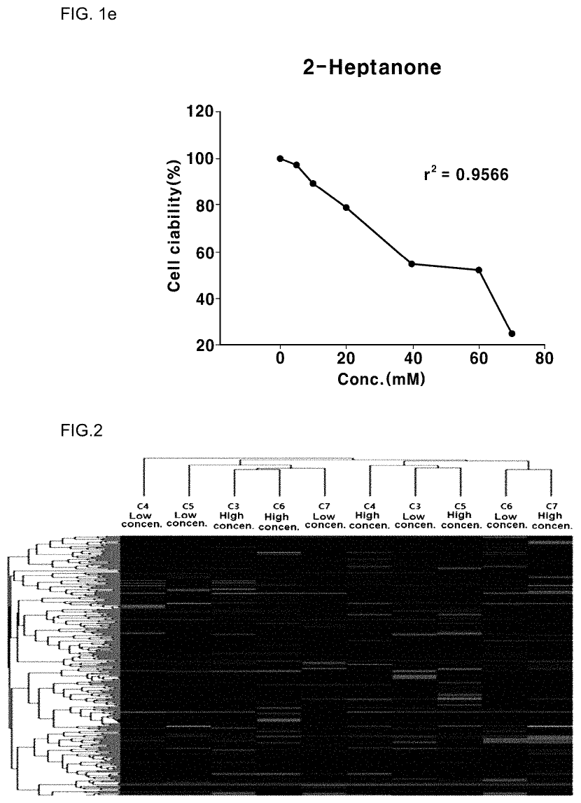

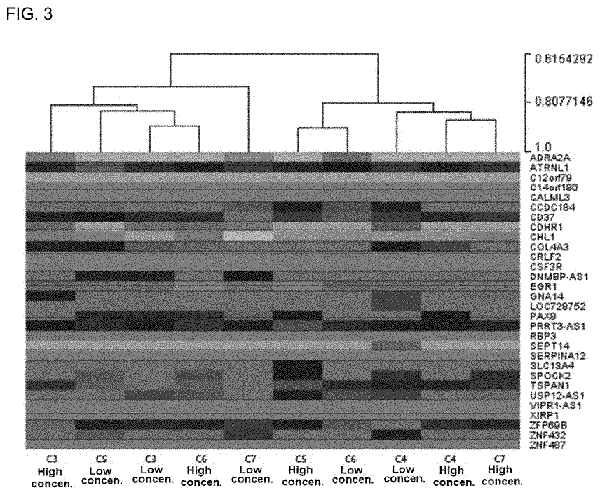

In one embodiment of the present invention, the present inventors treated five kinds of ketones including acetone, butanone, pentanone, hexanone and heptanone with a BEAS-2B cell line as a human bronchial epithelial cell line in order to find a biomarker for identifying exposure to ketones, to determine cytotoxicity. The result showed that the five types of ketones were toxic to the human bronchial epithelial cell line (see FIG. 1a to FIG. 1e), and the concentrations of the five ketones were determined based on the experiment. Then, the human bronchial epithelial cell line was treated at the determined concentration with the five ketones, and total RNA was isolated from the treated cell line. The isolated total RNA was converted to cDNA by oligo-dT primer hybridization and reverse transcription using a reverse transcriptase, and sequencing was then performed using NextSeq500 (Illumina, Inc., USA), a next-generation sequencing (NGS) instrument (see FIG. 2). The genes and expression levels of mRNAs were determined through sequencing, and 30 types of genes (15 types of over-expressed genes, shown in italic in Table 1, and 15 types of under-expressed genes, shown in bold in Table 1), expression of which is commonly changed by exposure of five ketones, among genes in which the expression rate of the experimental group and the control group differed by 2.0 times or more, were selected (see Table 1 and FIG. 3).

The 30 types of genes the expression of which was changed 2.0 times or more in common by exposure to the five ketone compounds were identified to be ADRA2A (adrenoceptor alpha 2A; GenBank NO. NM_000681), ATRNL1 (attractin-like 1; GenBank NO. NR_074088), LINC01619 (Synonyms:C12orf79, long intergenic non-protein coding RNA 1619; GenBank NO. NR_046159), C14orf180 (chromosome 14 open reading frame 180; GenBank NO. NM_001286399), CALML3 (calmodulin-like 3; GenBank NO. NM_005185), CCDC184 (coiled-coil domain containing 184; GenBank NO. NM_001013635), CD37 (CD37 molecule; GenBank NO. NM_001040031), CDHR (cadherin-related family member 1; GenBank NO. NM_001171971), CHL1 (cell adhesion molecule L1-like; GenBank NO. NM_001253387), COL4A3 (collagen type IV alpha 3; GenBank NO. NM_000091), CRLF2 (cytokine receptor-like factor 2; GenBank NO. NM_022148), CSF3R (colony-stimulating factor 3 receptor; GenBank NO. NM_172313), DNMBP-AS1 (DNMBP antisense RNA 1; GenBank NO. NR_024130), EGR1 (early growth response 1; GenBank NO. NM_001964), GNA14 (G protein subunit alpha 14; GenBank NO. NM_004297), LOC728752 (uncharacterized LOC728752; GenBank NO. NR_036504), PAX8 (paired box 8; GenBank NO. NM_003466), PRRT3-AS1 (PRRT3 antisense RNA 1; GenBank NO. NR_046734), RBP3 (retinol-binding protein 3; GenBank NO. NM_002900), SEPT14 (septin 14; GenBank NO. NM_207366), SERPINA12 (serpin family A member 12; GenBank NO. NM_001304461), SLC13A4 (solute carrier family 13 member 4; GenBank NO. NM_012450), SPOCK2 (sparc/osteonectin, cwcv and kazal-like domains proteoglycan (testican) 2; GenBank NO. NM_001244950), TSPAN1 (tetraspanin 1; GenBank NO. NM_005727), USP12-AS1 (USP12 antisense RNA 1; GenBank NO. NR_046547), VIPR1-AS1 (VIPR1 antisense RNA 1; GenBank NO. NR_046654), XIRP1 (xin actin binding repeat containing 1; GenBank NO. NM_001198621), ZFP69B (ZFP69 zinc finger protein B; GenBank NO. NM_023070), ZNF432 (zinc finger protein 432; GenBank NO. NM_014650), ZNF487 (zinc finger protein 487; GenBank NO. NR_026693). It was identified that the 30 kinds of genes have not been reported to be related to toxicity in bronchial epithelial cells upon exposure to ketone compounds (see Example 2-3).

Then, the biomarker of the present invention is a gene, the expression of which is commonly increased or decreased by exposure to five ketones, which is useful as a means to determine identification of exposure to ketones, monitoring, determination of risk, and the mechanism of pulmonary toxicity caused by ketones.

In one aspect, the present invention provides a kit for identifying specific exposure to five kinds of ketones including acetone, butanone, pentanone, hexanone and heptanone, including an agent for measuring an expression level of a gene (ADRA2A, adrenoceptor alpha 2A) of Gene Accession No. NM_000681 (GenBank).

In addition, the kit for identifying specific exposure to five kinds of ketones according to the present invention may include, but is not limited to, an agent for measuring an expression level of a gene further selected from the following group:

Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

As used herein, the term “expression level” refers to a determined level of gene expression. The expression level is an absolute value, or one reference gene (e.g., a housekeeping gene), an average of two or more reference genes, or a calculated average expression value or a determined level of gene expression relative to other informative gene without using a reference sample. The expression level of a gene can be measured directly, for example, by obtaining a signal, the signal intensity of which correlates with the amount of mRNA transcript of the gene. The expression level can also be obtained by competitive reaction with regard to the reference sample.

In the present invention, the agent for measuring the expression level of the gene may be, but is not limited to, sense and antisense primers or probes that complementarily bind to the mRNA of the gene.

As used herein, the term “primer” refers to a short gene sequence that is a starting point for DNA synthesis and means an oligonucleotide synthesized for use in diagnosis, DNA sequencing and the like. The primers can be typically synthesized to a length of 15 to 30 base pairs, but may vary depending on the purpose of use, and may be modified by methylation, capping, or the like by a known method.

As used herein, the term “probe” refers to a nucleic acid capable of specifically binding to mRNA of several bases to several hundred bases in length, which is produced through enzymatic chemical separation purification or synthesis. Radioisotopes, enzymes, or phosphors can be labeled to identify the presence or absence of mRNA, and can be designed and modified by known methods.

The expression level of mRNA can be measured through one or more methods selected from the group consisting of next-generation sequencing (NGS), transcriptome sequencing (RNA-Seq), NanoString nCounter analysis, polymerase chain reaction (PCR), reverse transcription polymerase chain reaction (RT-PCR), real-time PCR, RNase protection assay (RPA), microarray, and northern blotting.

Meanwhile, the kit of the present invention may further include at least one of a reaction reagent, a labeling reagent or a wash buffer, the reaction reagent may be selected from the group consisting of a buffer used for hybridization, oligo (dt) beads for separating mRNA from total RNA, a reverse transcriptase for synthesizing cDNA from RNA, dNTPs, rNTP (premixed or separately fed), a ligase and a combination thereof, but is not limited thereto, and may include all reaction reagents required for mRNA hybridization for RNA-sequencing (RNA-seq), cDNA synthesis and amplification reactions known to those skilled in the art.

In addition, the kit according to the present invention may further include human bronchial epithelial cells. The human bronchial epithelial cells are preferably BEAS-2B, but are not limited thereto. Any human bronchial epithelial cells may be used, as long as they are cells derived from human bronchus or human bronchial alveolar cell carcinoma and tissue.

One aspect of the present invention provides a method for identifying exposure to five ketones including acetone, butanone, pentanone, hexanone and heptanone, including the following steps:

a) isolating total RNA from somatic cells of each of experimental and normal control groups suspected of exposure to five ketones including acetone, butanone, pentanone, hexanone and heptanone;

b) converting the total RNA of the experimental and control groups of step a) to cDNA by treatment with oligo-dT primers and reverse transcriptase to establish a cDNA library;

c) sequencing the cDNA library obtained in step b) through high-throughput sequencing using next-generation sequencing (NGS) equipment; and

d) determining an expression level of genes included in the composition according to the present invention in the analyzed data of step c) compared to a control group.

In the method for identifying the exposure, the somatic cells of step a) are preferably human bronchial epithelial cells of a BEAS-2B cell line, but are not limited thereto, and any cell derived from human bronchus or human bronchial alveolar cell carcinoma and tissue. Preferably, the somatic cells may be bronchial epithelial cells obtained from a subject suspected of exposure to five ketones (experimental group) or a subject unexposed thereto (normal control group).

In the method of identifying exposure, the total RNA of step b) may be isolated using TRIzol Reagent, but is not limited thereto, and any total RNA purification and separation method known to those skilled in the art may be used.

In addition, the total RNA of step b) can be converted to cDNA through hybridization with oligo-dT primers and treatment with a reverse transcriptase, but the present invention is not limited thereto, and any method of converting total RNA to cDNA known to those skilled in the art may be used.

In the method of identifying exposure, the sequencing of step c) can be carried out through high-throughput sequencing using next-generation sequencing (NGS) equipment as a sequencing tool, but the present invention is not limited thereto, and any method known to those skilled in the art may be applied as long as it enables sequencing.

In the method of identifying exposure, the expression levels of the genes of step d) can be compared using GeneSpring GX 12.6.1 software (Agilent, USA), but the present invention is not limited thereto and any analysis software known to those skilled in the art may be used.

In one embodiment of the present invention, sequencing is carried out by high-through sequencing with single-end 75 sequencing using a NextSeq500 (Illumina, Inc., USA) as a next-generation sequencing (NGS) instrument. Read count (RC) data were processed based on a quantile normalization method using EdgeR in R (R development Core Team, 2016) using a Bioconductor (Gentleman et al., 2004). Gene classification was conducted based on searches conducted on DAVID (http://david.abcc.ncifcrf.gov/) and the Medline database (http://www.ncbi.nlm.nih.gov/). The extracted data was analyzed through normalization using Agilent GeneSpring GX 12.6.1 (Agilent technologies, CA, USA) to comparatively analyze the expression pattern of each gene (see Example 2-3).

The method for comparing and identifying a gene expression degree according to the present invention is ultimately useful as a means to determine identification of exposure to ketones, monitoring, determination of harmfulness, and the mechanism of pulmonary toxicity caused by ketones using high-through sequencing capable of identifying a distribution of genes specifically overexpressed or underexpressed due to exposure to ketones.

In addition, the present invention provides a microarray chip for specifically identifying exposure to five ketones including acetone, butanone, pentanone, hexanone and heptanone, wherein a nucleic acid sequence of any one or more genes selected from the following group or a complementary strand molecule thereof is integrated:

Gene Accession No. (GenBank) NM_000681 (ADRA2A, adrenoceptor alpha 2A), Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

The microarray chip for specifically identifying exposure to five ketones including acetone, butanone, pentanone, hexanone and heptanone according to the present invention can be produced by a method known to those skilled in the art. As an example, a method of producing the microarray chip is as follows. In order to immobilize the selected biomarker gene as a probe on a substrate of a microarray chip, a micropipetting method using a piezoelectric method, a method using a pin-type spotter or the like may be used, but the present invention is not limited thereto. The substrate of the microarray chip is preferably coated with one active group selected from the group consisting of amino-silane, poly-L-lysine and aldehyde. In addition, the substrate may be selected from the group consisting of a slide glass, a plastic, a metal, a silicone, a nylon film and a nitrocellulose film, but is not limited thereto.

By comparatively analyzing the expression changes of the marker genes in the cells of a subject and a control group using a microarray chip which probes DNA, cDNA, RNA and complementary strand molecules thereof containing all or part of the gene sequence, whether or not the subject was exposed to five types of ketones including acetone, butanone, pentanone, hexanone and heptanone can be identified.

In addition, the present invention provides a method for identifying exposure to the five types of ketones including analyzing the expression changes of marker genes in the cells of a subject using a microarray chip produced by probing DNA, cDNA, RNA of genes of the biomarker according to the present invention and complementary strand molecules thereof. That is, as described above, whether or not the subject is exposed to the five ketones can be identified by analyzing whether or not the marker genes are overexpressed or underexpressed in the cells of the subject, making it possible to determine whether or not the subject is exposed to the five ketones.

Hereinafter, the present invention will be described in more detail with reference to specific examples. However, the following examples are provided only for illustration of the present invention, and should not be construed as limiting the scope of the present invention. However, it will be obvious to those skilled in the art that the present invention is not limited to the following examples and a variety of modifications and alterations are possible without departing from the ideas and scope of the present invention.

### Example 1: Cell Culture and Chemical Treatment

### Example 1-1: Cell Culture

BEAS-2B (Korean Cell Line Bank), a human bronchial epithelial cell line, was cultured using RPMI 1640 medium (Gibco, USA) supplemented with 10% FBS until the cells grew to about a confluence of 70 to 80% in a 100 mm dish.

### Example 1-2: Selection of Ketone Compounds

Among ketones belonging to mVOCs (microbial volatile organic compounds) exposed to the environment, acetone, 2-butanone, 2-pentanone, 2-hexanone and 2-heptanone were selected, and the chemicals were dissolved in filtered distilled water (DW). A vehicle concentration was set to 0.1% or less in all experiments.

### Example 1-3: Selection of Exposure Concentration and Chemical Treatment by MTT Assay

MTT assay using a BEAS-2B cell line was performed in accordance with the method of Mossman et al. (J. Immunol. Methods, 65, 55-63, 1983).

Specifically, the cells were seeded at a density of 3.5×10⁴ cells/well in a 24-well plate and treated with the five ketone compounds dissolved in filtered distilled water, prepared in Example 1-2. After 48 hours, the result was mixed with 5 mg/ml of MTT (3-4,5-dimethylthiazol-2,5-diphenyltetrazolium bromide), added to a tube and incubated at 37° C. for 3 hours. Then, the medium was removed, the formed formazan crystal was dissolved in 500 μl of DMSO, and then 100 μl of the solution was transferred to a 96-well plate to measure an OD (optical density) value at 540 nm.

As a result, as shown in FIG. 1a to FIG. 1e, the cytotoxicity of the ketone compounds in the BEAS-2B cell line was determined. As a result, the concentration (105) providing a survival rate (viability) of 95% was 5.38 mM for acetone, 22.27 mM for butanone, 5.24 mM for pentanone, 14.82 mM for hexanone and 5.05 mM for heptanone, and the concentration (1020) providing a survival rate (viability) of 80% was 71.90 mM for acetone, 106.21 mM for butanone, 39.33 mM for pentanone, 52.51 mM for hexanone, and 20.22 mM for heptanone (FIG. 1a to FIG. 1e).

### Example 2: Gene-Sequencing Experiment

Gene-sequencing experiments were performed based on the results of the cytotoxicity test of ketone compounds in BEAS-2B, the human bronchial epithelial cell line in Examples 1-3.

### Example 2-1: Isolation of RNA

RNA was extracted from cell samples of ketone-exposed and non-ketone-exposed groups using a TRIzol reagent (Life Technologies, USA) in accordance with the manufacturer's directions to isolate total RNA and the total RNA was purified using a RNeasy mini kit (Qiagen, USA). The concentration of each total RNA sample was determined by a ND-2000 spectrophotometer (Thermo Inc., DE, USA) as a spectrophotometer and Agilent 2100 Bioanalyzer (Agilent Technologies, USA).

### Example 2-2: Library Preparation

The establishment of libraries from the total RNA obtained in Example 2-1 in the experimental group and the normal control group exposed to the ketones was carried out using a QuantSeq 3′ mRNA-Seq Library Prep Kit (Lexogen, Inc., Austria) in accordance with the manufacturer's directions. 500 ng of the total RNA was hybridized with an oligo-dT primer including an Illumina-compatible sequence at the 5′ end of RNA, followed by reverse transcription. After degradation of the RNA template, a second strand was synthesized using a random primer including an Illumina-compatible linker sequence at the 5′ end thereof. Double-stranded libraries were purified using magnetic beads to remove all reaction components. The library was amplified in order to add the entire adapter sequence required for cluster production, and the completed cDNA library was purified from the PCR components.

### Example 2-3: Sequencing

Sequencing was performed by high-throughput sequencing as single-end 75 sequencing using a NextSeq 500 (IIlumina, Inc., USA). Read count (RC) data was processed based on the quantile normalization method using EdgeR in R (R development Core Team, 2016) using Bioconductor (Gentleman et al., 2004). Gene classification was based on searches performed on DAVID (http://david.abcc.ncifcrf.gov/) and the Medline database (http://www.ncbi.nlm.nih.gov/).

The extracted data were analyzed through normalization using Agilent GeneSpring GX 12.6.1 (Agilent technologies, CA, USA) to analyze the expression pattern of each gene.

As a result, as can be seen from Table 1 and FIG. 3, the genes, the expression of which was changed 2.0 times or more in common upon exposure to five kinds of ketone compounds, were identified to be 30 types. Specifically, 30 types of genes, the expression of which was changed 2.0 times or more in common upon exposure to the five kinds of ketones, that is, acetone, 2-butanone, 2-pentanone, 2-hexanone and 2-heptanone, at concentrations corresponding to IC₅ and IC₂₀, were ADRA2A (adrenoceptor alpha 2A; GenBank No. NM_000681), ATRNL1 (attractin-like 1; GenBank No. NR_074088), LINC01619 (Synonyms: C12orf79, long intergenic non-protein coding RNA 1619; GenBank No. NR_046159), C14orf180 (chromosome 14 open reading frame 180; GenBank No. NM_001286399), CALML3 (calmodulin-like 3; GenBank No. NM_005185), CCDC184 (coiled-coil domain containing 184; GenBank No. NM_001013635), CD37 (CD37 molecule; GenBank No. NM_001040031), CDHR (cadherin-related family member 1; GenBank No. NM_001171971), CHL1 (cell adhesion molecule L1-like; GenBank No. NM_001253387), COL4A3 (collagen type IV alpha 3; GenBank No. NM_000091), CRLF2 (cytokine receptor-like factor 2; GenBank No. NM_022148), CSF3R (colony-stimulating factor 3 receptor; GenBank No. NM_172313), DNMBP-AS1 (DNMBP antisense RNA 1; GenBank No. NR_024130), EGR1 (early growth response 1; GenBank No. NM_001964), GNA14 (G protein subunit alpha 14; GenBank No. NM_004297), LOC728752 (uncharacterized LOC728752; GenBank No. NR_036504), PAX8 (paired box 8; GenBank No. NM_003466), PRRT3-AS1 (PRRT3 antisense RNA 1; GenBank No. NR_046734), RBP3 (retinol-binding protein 3; GenBank No. NM_002900), SEPT14 (septin 14; GenBank No. NM_207366), SERPINA12 (serpin family A member 12; GenBank No. NM_001304461), SLC13A4 (solute carrier family 13 member 4; GenBank No. NM_012450), SPOCK2 (sparc/osteonectin, cwcv and kazal-like domains proteoglycan (testican) 2; GenBank No. NM_001244950), TSPAN1 (tetraspanin 1; GenBank No. NM_005727), USP12-AS1 (USP12 antisense RNA 1; GenBank No. NR_046547), VIPR1-AS1 (VIPR1 antisense RNA 1; GenBank No. NR_046654), XIRP1 (xin actin binding repeat containing 1; GenBank No. NM_001198621), ZFP69B (ZFP69 zinc finger protein B; GenBank No. NM_023070), ZNF432 (zinc finger protein 432; GenBank No. NM_014650), and ZNF487 (zinc finger protein 487; GenBank No. NR_026693). Meanwhile, it was identified that 30 types of genes have not been reported to be associated with toxicity in bronchial epithelial cells upon exposure to ketone compounds.

TABLE 1
Genes, the expression of which is changed in common upon exposure to five kinds of ketone

| | | Fold change | | | | | | | | | |
|---|---|---|---|---|---|---|---|---|---|---|---|
| | | | | 2- | 2- | 2- | 2- | 2- | 2- | 2- | 2- |
| | | Ace- | Ace- | Buta- | Buta- | Penta- | Penta- | Hexa- | Hexa- | Hepta- | Hepta- |
| | | tone | tone | none | none | none | none | none | none | none | none |
| Accession | Name of | 5.38 | 71.90 | 22.27 | 106.21 | 5.24 | 39.38 | 14.82 | 52.51 | 5.05 | 20.22 |
| No. | gene | mM | mM | mM | mM | mM | mM | mM | mM | mM | mM |
| NM_000681 | ADRA2A | 0.179 | 0.355 | 0.185 | 0.184 | 0.179 | 0.184 | 0.410 | 0.179 | 0.321 | 0.179 |
| NR_074088 | ATRNL1 | 3.000 | 2.751 | 3.234 | 2.330 | 4.158 | 2.665 | 2.058 | 2.281 | 3.365 | 3.204 |
| NR_046159 | C12orf79 | 0.213 | 0.213 | 0.216 | 0.216 | 0.213 | 0.216 | 0.218 | 0.213 | 0.213 | 0.213 |
| NR_046159 | C14orf80 | 0.354 | 0.354 | 0.356 | 0.356 | 0.354 | 0.356 | 0.358 | 0.354 | 0.354 | 0.354 |
| NM_001286399 | CALML3 | 0.354 | 0.354 | 0.356 | 0.356 | 0.354 | 0.356 | 0.358 | 0.354 | 0.354 | 0.354 |
| NM_001013635 | CCDC184 | 12.166 | 4.927 | 2.379 | 6.647 | 4.501 | 2.387 | 3.872 | 6.982 | 4.098 | 6.381 |
| NM_001040031 | CD37 | 2.781 | 2.526 | 3.286 | 2.647 | 2.028 | 3.272 | 4.214 | 2.819 | 5.216 | 3.227 |
| NM_001171971 | CDHR1 | 0.409 | 0.429 | 0.470 | 0.222 | 0.216 | 0.223 | 0.228 | 0.432 | 0.386 | 0.216 |
| NM_001253387 | CHL1 | 0.224 | 0.360 | 0.274 | 0.268 | 0.335 | 0.273 | 0.296 | 0.364 | 0.122 | 0.335 |
| NM_000091 | COL4A3 | 4.046 | 2.490 | 2.264 | 3.577 | 2.229 | 5.033 | 4.765 | 4.424 | 4.774 | 8.193 |
| NM_022148 | CRLF2 | 0.355 | 0.357 | 0.359 | 0.359 | 0.355 | 0.359 | 0.362 | 0.355 | 0.355 | 0.355 |
| NM_172313 | CSF3R | 0.354 | 0.358 | 0.356 | 0.356 | 0.354 | 0.356 | 0.358 | 0.354 | 0.354 | 0.354 |
| NR_024130 | DNMBP-AS1 | 2.455 | 5.285 | 11.453 | 12.732 | 2.406 | 8.289 | 14.919 | 5.009 | 2.035 | 11.098 |
| NM_001964 | EGR1 | 0.422 | 0.437 | 0.422 | 0.404 | 0.419 | 0.332 | 0.448 | 0.328 | 0.410 | 0.381 |
| NM_004297 | GNA14 | 5.492 | 2.289 | 3.512 | 5.582 | 8.261 | 6.776 | 8.515 | 4.986 | 4.971 | 4.498 |
| NR_036504 | LOC728752 | 4.554 | 4.939 | 3.506 | 6.647 | 7.322 | 5.683 | 6.212 | 6.982 | 5.873 | 5.440 |
| NM_003466 | PAX8 | 2.753 | 5.800 | 6.636 | 2.233 | 2.723 | 2.277 | 5.964 | 2.992 | 4.275 | 4.602 |
| NR_046734 | PRRT3-AS1 | 2.261 | 2.061 | 2.207 | 2.147 | 2.817 | 3.902 | 2.811 | 3.123 | 2.285 | 2.525 |
| NM_002900 | RBP3 | 0.354 | 0.376 | 0.356 | 0.356 | 0.354 | 0.356 | 0.358 | 0.354 | 0.354 | 0.354 |
| NM_207366 | SEPT14 | 0.214 | 0.214 | 0.458 | 0.217 | 0.214 | 0.218 | 0.220 | 0.214 | 0.214 | 0.214 |
| NM_001304461 | SERPINA12 | 0.354 | 0.354 | 0.356 | 0.356 | 0.354 | 0.356 | 0.358 | 0.354 | 0.354 | 0.354 |
| NM_012450 | SLC13A4 | 10.528 | 12.386 | 7.697 | 8.571 | 8.995 | 2.153 | 4.947 | 7.012 | 4.638 | 11.825 |
| NM_001244950 | SPOCK2 | 5.291 | 5.054 | 3.020 | 8.426 | 4.031 | 2.181 | 4.851 | 3.833 | 4.800 | 2.838 |
| NM_005727 | TSPAN1 | 5.935 | 2.949 | 2.540 | 2.466 | 4.281 | 3.938 | 2.828 | 4.272 | 4.660 | 3.058 |
| NR_046647 | USP12-AB1 | 3.620 | 6.910 | 3.499 | 5.570 | 7.329 | 2.362 | 3.862 | 3.988 | 11.319 | 4.509 |
| NR_046654 | VIPR†-AS1 | 0.354 | 0.354 | 0.356 | 0.356 | 0.354 | 0.356 | 0.358 | 0.354 | 0.354 | 0.354 |
| NM_001198621 | XIRP1 | 0.354 | 0.354 | 0.356 | 0.356 | 0.354 | 0.356 | 0.358 | 0.354 | 0.354 | 0.354 |
| NM_023070 | ZFP69B | 2.708 | 4.454 | 4.676 | 2.942 | 2.472 | 2.299 | 4.095 | 2.573 | 3.346 | 2.674 |
| NM_014650 | ZNF432 | 4.647 | 5.319 | 2.326 | 6.342 | 4.086 | 5.290 | 5.206 | 4.577 | 3.207 | 4.845 |
| NR_026693 | ZNF487 | 0.354 | 0.354 | 0.356 | 0.356 | 0.354 | 0.356 | 0.358 | 0.354 | 0.354 | 0.354 |

As apparent from the foregoing, fast-throughput sequencing using, as a biomarker, a gene showing a changed expression pattern upon exposure to five types of ketones including acetone, butanone, pentanone, hexanone and heptanone according to an aspect of the present invention has the effects of monitoring ketones, and quickly and accurately determining the risk of ketones.

In addition, the biomarker and the method of identifying the exposure of ketones using the biomarker are useful as a means for identifying the specific risk action mechanism of caused by ketones.

The effects of the present invention are not limited to those mentioned above. It should be understood that the effects of the present invention include all effects that can be inferred from the description of the present invention.

The invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

1. A biomarker composition for identifying specific exposure to five types of ketones comprising acetone, butanone (2-butanone), pentanone (2-pentanone), hexanone (2-hexanone) and heptanone (2-heptanone), comprising an ADRA2A (adrenoceptor alpha 2A) gene of Gene Accession No. NM_000681 (GenBank).

2. The biomarker composition according to claim 1, wherein the biomarker composition comprises one or more of genes further selected from the following group:
Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

3. The biomarker composition according to claim 1, wherein expression of the ADRA2A (adrenoceptor alpha 2A) gene of Gene Accession No. NM_000681 (GenBank) is increased or decreased by exposure to five kinds of ketones comprising acetone, butanone, pentanone, hexanone and heptanone.

4. A kit for identifying specific exposure to five kinds of ketones comprising acetone, butanone, pentanone, hexanone and heptanone, the kit comprising an agent for measuring an expression level of a gene ADRA2A (adrenoceptor alpha 2A) of Gene Accession No. NM_000681 (GenBank).

5. The kit according to claim 4, wherein the kit further comprises an agent for measuring an expression level of a gene further selected from the following group:
Gene Accession No. (GenBank) NR_074088 (ATRNL1, attractin-like 1), Gene Accession No. (GenBank) NR_046159 (LINC01619; C12orf79, long intergenic non-protein coding RNA 1619), Gene Accession No. (GenBank) NM_001286399 (C14orf180, chromosome 14 open reading frame 180), Gene Accession No. (GenBank) NM_005185 (CALML3, calmodulin-like 3), Gene Accession No. (GenBank) NM_001013635 (CCDC184, coiled-coil domain containing 184), Gene Accession No. (GenBank) NM_001040031 (CD37, CD37 molecule), Gene Accession No. (GenBank) NM_001171971 (CDHR1, cadherin-related family member 1), Gene Accession No. (GenBank) NM_001253387 (CHL1, cell adhesion molecule L1-like), Gene Accession No. (GenBank) NM_000091 (COL4A3, collagen type IV alpha 3), Gene Accession No. (GenBank) NM_022148 (CRLF2, cytokine receptor-like factor 2), Gene Accession No. (GenBank) NM_172313 (CSF3R, colony-stimulating factor 3 receptor), Gene Accession No. (GenBank) NR_024130 (DNMBP-AS1, DNMBP antisense RNA 1), Gene Accession No. (GenBank) NM_001964 (EGR1, early growth response 1), Gene Accession No. (GenBank) NM_004297 (GNA14, G protein subunit alpha 14), Gene Accession No. (GenBank) NR_036504 (LOC728752, uncharacterized LOC728752), Gene Accession No. (GenBank) NM_003466 (PAX8, paired box 8), Gene Accession No. (GenBank) NR_046734 (PRRT3-AS1, PRRT3 antisense RNA 1), Gene Accession No. (GenBank) NM_002900 (RBP3, retinol-binding protein 3), Gene Accession No. (GenBank) NM_207366 (SEPT14, septin 14), Gene Accession No. (GenBank) NM_001304461 (SERPINA12, serpin family A member 12), Gene Accession No. (GenBank) NM_012450 (SLC13A4, solute carrier family 13 member 4), Gene Accession No. (GenBank) NM_001244950 (SPOCK2, sparc/osteonectin, cwcv and kazal-like domains proteoglycan (testican) 2), Gene Accession No. (GenBank) NM_005727 (TSPAN1, tetraspanin 1), Gene Accession No. (GenBank) NR_046547 (USP12-AS1, USP12 antisense RNA 1), Gene Accession No. (GenBank) NR_046654 (VIPR1-AS1, VIPR1 antisense RNA 1), Gene Accession No. (GenBank) NM_001198621 (XIRP1, xin actin binding repeat containing 1), Gene Accession No. (GenBank) NM_023070 (ZFP69B, ZFP69 zinc finger protein B), Gene Accession No. (GenBank) NM_014650 (ZNF432, zinc finger protein 432), and Gene Accession No. (GenBank) NR_026693 (ZNF487, zinc finger protein 487).

6. The kit according to claim 4, wherein the kit comprises at least one of a reaction reagent, a labeling reagent or a wash buffer,
wherein the reaction reagent is selected from the group consisting of a buffer used for hybridization, oligo (dt) beads for separating mRNA from total RNA, a reverse transcriptase for synthesizing cDNA from RNA, dNTPs, rNTP (premixed or separately fed), a ligase and a combination thereof.

7. The kit according to claim 4, wherein the kit further comprises human bronchial epithelial cells.

8. The kit according to claim 4, wherein the agent for measuring the expression level of the gene comprises sense and antisense primers or probes that complementarily bind to mRNA of the gene.

9. A method for identifying exposure to five ketones comprising acetone, butanone, pentanone, hexanone and heptanone, comprising:
a) isolating total RNA from somatic cells of each of experimental and normal control groups suspected of exposure to five ketones comprising acetone, butanone, pentanone, hexanone and heptanone;
b) converting the total RNA of the experimental and control groups of step a) to cDNA by treatment with oligo-dT primers and reverse transcriptase to establish a cDNA library;
c) sequencing the cDNA library obtained in step b) through high-throughput sequencing using next-generation sequencing (NGS) equipment; and
d) determining an expression level of genes included in the composition of claim 1 in the analyzed data of step c) compared to a control group.

10. The method according to claim 9, wherein the somatic cells of step a) comprise human bronchial epithelial cells.

11. The method according to claim 9, wherein the human bronchial epithelial cells comprise BEAS-2B cells.

12. A method for identifying exposure to five ketones comprising acetone, butanone, pentanone, hexanone and heptanone, comprising:
a) isolating total RNA from somatic cells of each of experimental and normal control groups suspected of exposure to five ketones comprising acetone, butanone, pentanone, hexanone and heptanone;
b) converting the total RNA of the experimental and control groups of step a) to cDNA by treatment with oligo-dT primers and reverse transcriptase to establish a cDNA library;
c) sequencing the cDNA library obtained in step b) through high-throughput sequencing using next-generation sequencing (NGS) equipment; and
d) determining an expression level of genes included in the composition of claim 2 in the analyzed data of step c) compared to a control group.

13. The method according to claim 12, wherein the somatic cells of step a) comprise human bronchial epithelial cells.

14. The method according to claim 12, wherein the human bronchial epithelial cells comprise BEAS-2B cells.
